(19) United States
(12) Patent Application Publication
(10) Pub. No.: US 2010/0063384 A1
(43) Pub. Date: Mar. 11, 2010

(54) LOCAL INTRA-BODY DELIVERY SYSTEM

(75) Inventors: Giora Kornblau, Binyamina (IL); David M. Neustadter, Doar-Na Shimshon - Moshav Nof Ayalon (IL); Tal Shchory, Yokneam (IL); Saul Stokar, RaAnana (IL)

Correspondence Address: MARTIN D. MOYNIHAN d/b/a PRTSI, INC., P.O. BOX 16446, ARLINGTON, VA, 22215, US

(73) Assignee: Navotek Medical Ltd., Yokneam (IL)

(21) Appl. No.: 12/312,504

(22) PCT Filed: Nov. 14, 2007
(86) PCT No.: PCT/IL2007/001406

§ 371 Date: May 14, 2009

Related U.S. Application Data
(60) Provisional application No. 60/865,888, filed on Nov. 15, 2006.

(51) Int. Cl. A61B 5/0275; A61B 5/06; A61M 37/00

(52) U.S. Cl. 600/424; 600/505; 604/502

(57) ABSTRACT

A system for delivery of a capsule to a target location within a subject body including a capsule including a locomotion element and a gamma emitting radioactive source, a radiation tracking subsystem capable of locating the gamma emitting radioactive source in three dimensions, and a locomotion control subsystem capable of controlling movement of the capsule by effecting movement of the locomotion element, based, at least partly, on a location of the gamma emitting radioactive source provided by the radiation tracking subsystem. A method of measuring a velocity of flow of a fluid at a target location within a subject body including inserting a capsule including a locomotion element and a gamma emitting radioactive source into the body, using a radiation tracking subsystem to locate the gamma emitting radioactive source in three dimensions, moving the capsule to the target location within the body using a locomotion control subsystem which controls movement of the capsule by effecting movement of the locomotion element, based, at least partly, on location of the gamma emitting radioactive source provided by the radiation tracking subsystem, and measuring the velocity of flow of the fluid at the target location. Related apparatus and methods are also described.

### FIELD OF THE INVENTION

The present invention relates to local delivery of an object within a body, and more, particularly, but not exclusively, to a system and method for placement of an intra-body drug delivery mechanism at an appropriate location within a body channel, and for measurement of body fluid flow using the intra-body drug delivery system. The invention further relates non-exclusively to an automatic system and method for placement of the object at an appropriate location within a body channel.

### BACKGROUND OF THE INVENTION

There are numerous potential medical applications for local intra-body object delivery. Some examples include local delivery of thrombolytic agents to a site of thrombosis, and local delivery of chemotherapeutic agents to tumors. Such techniques are beginning to be used, with a drug being delivered through intra-body catheters. However, in some applications, particularly in the brain, the use of the catheter techniques is severely limited by a level of expertise required to perform the catheterization procedure. In addition, for emergency applications such as stroke, the need for a catheterization lab can also limit the use of this procedure.

A system that uses external magnets and a specialized catheter tip to aid in the catheterization process, reducing the level of expertise needed on part of an interventional radiologist, is described in U.S. Pat. No. 7,066,924 to Garibaldi et al. However, this system still requires fluoroscopic guidance and an involvement of the interventional radiologist.

A system for magnetic orienting and maneuvering of a magnetic element within a body structure is described in U.S. Pat. No. 6,292,678 to Hall et al.

A system for magnetic orienting of a catheter tip within arteries is produced by Stereotaxis Inc., of St. Louis, Mo., USA, and a general description thereof is available on the World Wide Web at, for example, www.stereotaxis.com/Products-Technology/Magnetic-Navigation/. Additional details, including the mathematics that explains how the field gradient exerts a force, can be found in a Master's thesis by Jeffery Leach (Virginia Polytechnic Institute and State University, 2003) found on the World Wide Web at scholarlib.vt.edu/theses/available/etd-02182003-085930/unrestricted/ETD.pdf.

A need for fluoroscopic guidance can be eliminated by replacing it with three dimensional tracking of the tip of the catheter integrated with a three dimensional angiographic dataset based on CT, MRI, or 3D angiography. The combination of 3D tracking and catheter navigation/steering has been suggested in U.S. Provisional Patent Application 60/619,792, which is incorporated into PCT Published Patent Application WO 2006/016368 of Navotek Medical Ltd., and into US Published Patent Application 2007/205373 of Kornblau et al. The disclosures of the patent applications mentioned above are hereby incorporated herein by reference.

Much of the complication of micro-catheterization, particularly in the brain, results from the need for prior art catheters to be rigid enough to retain their form as they are pushed through vasculature, yet flexible enough to bend around branching vasculature without damaging the blood vessels. The ideal mechanical properties for a catheter depend on whether the catheter is inserted directly into the vasculature or along a guide wire, what region of the vasculature it is used for, and what application it is used for, but all catheters that are pushed through the vasculature need a level of rigidity that makes maneuvering through the complex vasculature of the brain difficult.

One method of positioning an electrode in the brain is by using a needle to insert the probe through suitable parts of the brain. For example, an electrode is inserted into the hypothalamus, using a needle, to treat Parkinson's disease. The treatment is known as deep brain stimulation.

The disclosures of all references mentioned above and throughout the present specification, as well as the disclosures of all references mentioned in those references, are hereby incorporated herein by reference.

### SUMMARY OF THE INVENTION

The present invention seeks to provide an improved system and method for placement of an object at an appropriate location within a body. More particularly, but not exclusively, a system and method for placement of an intra-body substance delivery mechanism at an appropriate location within a body channel, and for measurement of body fluid flow using the intra-body substance delivery system. The invention further comprises an automatic system and method for placement of the object at an appropriate location within a body channel.

One exemplary embodiment of an object placed by the object placement system is an intra-body drug delivery mechanism.

There is a need for an intra-body drug delivery system that either eliminates the need for a catheter altogether or at least eliminates the need for catheter rigidity. Both of these possibilities are achieved by a Micro-Vascular-Capsule (MVC) which can be navigated within arteries. The capsule can either contain a drug within it or pull a flexible tube into the vasculature, to be used for drug delivery in place of a prior art semi-rigid catheter. A micro-vascular-capsule navigation system and a flexible tube pulling system are embodiments of this invention.

Some embodiments of the present inventions comprise object placement at a target location by navigating the object through blood vessels.

Some embodiments of the present inventions comprise object placement at a target location by navigating the object through lymph channels.

Some embodiments of the present inventions comprise object placement at a target location by navigating the object through channels comprising Cerebro-Spinal Fluid, such as along the spine.

Some embodiments of the present inventions comprise object placement at a target location within a patient's brain.

Some exemplary embodiments comprising navigating through a brain involve propulsion of the object, as described below for other embodiments, yet through brain tissue itself, and not necessarily through fluid channels within the brain.

Exemplary embodiments of the present invention will be described in the context of an intra-body delivery system for delivering a substance through blood vessels, and more specifically through arteries. The exemplary embodiments are to be understood as pertaining also to other body channels such as, for example, the body channels mentioned above.

Some embodiments of the present invention include an MVC which can be inserted into an artery and navigated to a particular location for local drug delivery. Position tracking for the MVC is based on a radiation tracking subsystem, such as described in PCT Published Patent Application WO 2006/016368 of Navotek Medical Ltd. and in PCT Published Patent Application WO 2007/017846 of Navotek Medical Ltd. The radiation tracking subsystem enables a tracked element to be small, for example approximately 0.01 mm³. By way of contrast, RF electromagnetic tracking typically requires a tracked element to be several millimeters in length. The small volume of the tracked element leaves more volume within the MVC for other components, potentially including but not limited to, a locomotion component and potentially for a drug payload to be released at a target location. For example, a capsule which is 0.3 mm in diameter and 2 mm long has a volume of approximately 0.14 mm³. In this case the radioactive tracked element occupies only 7% of the volume of the capsule. In addition, the small volume of the tracked element allows use of MVCs that are easy to introduce into a blood vessel, such as, for example, by being injected using a thin needle, and which are small enough to penetrate into very small blood vessels.

Some embodiments of the present invention perform placement of an intra-body drug delivery mechanism at an appropriate location within a blood vessel automatically, based on data such as a 3D angiography of a patient and location of the MVC.

In some embodiments of the present invention drug delivery is implemented by a triggered or time-release mechanism of a payload which is incorporated into the MVC. Alternatively, the drug can be delivered through a flexible tube which is pulled along into the artery by the MVC.

The term “drug” used throughout the present specification and claims is not intended to limit the invention to the delivery of any particular type of substance. The term drug is used throughout the present specification and claims interchangeably for any substance which is delivered to a target location using this invention.

In one embodiment, the payload in the MVC is the drug itself, which is released into the artery. In an alternative embodiment, the payload is a substance which is bound to the MVC, and which acts as a local activator for a substance which is delivered systemically. In this way, a small amount of substance incorporated into an MVC of small volume activates a large amount of the systemically delivered substance, transforming the systemically delivered substance into a therapeutic agent, while ensuring that the substance is activated only in a local region where it is needed. The concept of locally activated systemic drugs has been described in U.S. Pat. No. 6,569,688 to Sivan et al, which describes an activator being incorporated into an implanted carrier. An ability to place the activator automatically using an MVC instead of using an implanted carrier makes this type of drug treatment more accessible in applications in which there is no need for a stent or other permanent implant.

If a flexible tube is used for drug delivery, the tube optionally serves as a mechanical tether for the MVC. The tether aids in the navigation procedure by controlling forward motion of the MVC as the MVC is swept along by blood flow. If necessary, adhesion of the flexible tube to the walls of a blood vessel can be avoided by injecting bursts of fluid through the tube in order to cause it to vibrate. If a tube is not used, an anchoring mechanism is optionally built into the MVC to allow the MVC to anchor within the blood vessel at the treatment location.

Locomotion of the MVC can be achieved in a number of ways. Appropriate locomotion mechanisms include, but are not limited to, mechanical hydrodynamic steering and propulsion, such as propellers, fins, flagella, a swimming movement, and so on; mechanical movement along the walls of the vessels; and magnetic steering and propulsion using external magnets.

Some embodiments of the invention include an ability of the MVC to measure existence of blood flow and velocity of blood flow in the blood vessel in which it is placed. There are a number of ways in which this is achieved. For instance, measuring a time between changes in temperature or changes in impedance at the MVC after the release of a fluid from the MVC, or the attached flexible tube, can indicate blood flow and enable estimating the blood flow rate. Another embodiment releases a small amount of a radioactive liquid, such as Technetium-99m, into the blood around the MVC and measures the time taken for the radioactivity to diffuse using the sensors of the radiation tracking subsystem.

According to an aspect of some embodiments of the present invention there is provided a system for delivery of a capsule to a target location within a subject body including a capsule including a locomotion element and a gamma emitting radioactive source, a radiation tracking subsystem capable of locating the gamma emitting radioactive source in three dimensions, and a locomotion control subsystem capable of controlling movement of the capsule by effecting movement of the locomotion element, based, at least partly, on a location of the gamma emitting radioactive source provided by the radiation tracking subsystem.

According to some embodiments of the invention, the locomotion control subsystem is configured to use location information from the radiation tracking subsystem to automatically control movement of the capsule to the target location.

According to some embodiments of the invention, the locomotion control subsystem is configured to automatically control movement of the capsule from a first location in the subject body to the target location.

According to some embodiments of the invention, the locomotion control subsystem controls movement of the capsule to the target location is based, at least partly, on a three dimensional angiographic dataset.

Further according to some embodiments of the invention, including an optical tracker capable of monitoring the subject body and providing data for converting coordinates provided by the radiation tracking subsystem to coordinates provided by the three dimensional angiographic dataset.

According to some embodiments of the invention, the radioactive source emits gamma rays, the source having an activity between 0.001 mCi and 0.5 mCi. According to some embodiments of the invention, the gamma emitting radioactive source occupies less than 10% of the capsule's volume.

According to some embodiments of the invention, the locomotion element in the capsule includes a magnetic material and the locomotion control subsystem includes a magnetic field configured to apply to the magnetic material in the capsule at least one member of the group consisting of a force and a torque. According to some embodiments of the invention, the magnetic material includes a permanent magnet. According to some embodiments of the invention, the magnetic material includes a ferromagnet. According to some embodiments of the invention, the magnetic material includes a paramagnetic material.

Further according to some embodiments of the invention, including a substance delivery mechanism including a substance to be delivered and a release mechanism configured for releasing the substance.

According to some embodiments of the invention, the substance delivery mechanism includes a substance which is incorporated within the capsule.

Further according to some embodiments of the invention, the system configured to measure velocity of flow at the target location by releasing the substance and measuring dispersal of the substance.

According to some embodiments of the invention, the substance is radioactive and the radiation tracking subsystem is configured to measure dispersal of the radioactive substance, to measure a time taken for the radioactive substance to disperse, and to calculate the velocity of flow based, at least partly, on the time and the dispersal.

Further according to some embodiments of the invention, the system configured to measure impedance at the capsule, at two or more different times after the release of the substance, and configured to calculate the velocity of flow based, at least partly, on the measured impedances and the times the impedances were measured.

Further according to some embodiments of the invention, the system configured to measure temperature at the capsule, at two or more different times after the release of the substance, and to calculate the velocity of flow based, at least partly, on the measured temperatures and on the times the temperatures were measured.

According to some embodiments of the invention, the substance delivery mechanism includes a tube which is connected to the capsule through which a substance is delivered to the location of the capsule.

According to some embodiments of the invention, the locomotion control subsystem includes a tether connected to the capsule. According to some embodiments of the invention, the tether includes a tube capable of delivering a substance to a location of the capsule.

According to some embodiments of the invention, the locomotion control subsystem includes a tether control mechanism including one or more wheels and a spring mechanism, wherein at least one of the wheels is motorized, at least one of the wheels is in contact with the tube, and controls motion of the tube, at least one of the wheels is capable of being held against the tube by the spring mechanism, and when there is sufficient pressure within the tube, the tube is capable of opening enough to allow a substance within the tube to flow past the wheels.

According to some embodiments of the invention, the locomotion control subsystem includes a tether control mechanism including one or more wheels and a spring mechanism, wherein at least one of the wheels is motorized, at least one of the wheels is in contact with the tube, and controls motion of the tube, at least one of the wheels is in contact with the tube and includes a groove in contact with the tube, and when there is sufficient pressure within the tube, the tube is capable of stretching open slightly at a location of the groove, allowing a substance within the tube to flow past the wheels.

According to some embodiments of the invention, the tether includes at least one wire capable of conducting electrical current.

According to some embodiments of the invention, the system is configured to deliver the capsule to the target location through one or more body channels. According to some embodiments of the invention, the one or more body channels are blood vessels. According to some embodiments of the invention, the capsule is configured to receive locomotion from blood flow and the locomotion control subsystem is configured to provide steering to the capsule. According to some embodiments of the invention, the body channel is a lymph channel. According to some embodiments of the invention, the body channel includes a Cerebro-Spinal Fluid filled cavity.

According to some embodiments of the invention, the system is configured to deliver the capsule to the target location through brain tissue.

According to an aspect of some embodiments of the present invention there is provided a method of delivering a capsule to a target location within a subject body including inserting a capsule including a locomotion element and a gamma emitting radioactive source into the body, using a radiation tracking subsystem to locate the gamma emitting radioactive source in three dimensions, and moving the capsule to the target location within the body using a locomotion control subsystem to control movement of the capsule by effecting movement of the locomotion element, based, at least partly, on location of the gamma emitting radioactive source provided by the radiation tracking subsystem.

According to some embodiments of the invention, the locomotion control subsystem uses location information from the radiation tracking subsystem to automatically control movement of the capsule to the target location.

According to some embodiments of the invention, the locomotion control subsystem automatically controls movement of the capsule from a first location in the subject body to the target location.

According to some embodiments of the invention, the locomotion control subsystem controls movement of the capsule to the target location based, at least partly, on a three dimensional angiographic dataset.

Further according to some embodiments of the invention, including an optical tracker capable of monitoring the subject body and providing data for converting coordinates provided by the radiation tracking subsystem to coordinates provided by the three dimensional angiographic dataset.

According to some embodiments of the invention, the radioactive source emits gamma rays with an activity between 0.001 mCi and 0.5 mCi. According to some embodiments of the invention, the gamma emitting radioactive source occupies less than 10% of the capsule's volume.

Further according to some embodiments of the invention, releasing a substance from the capsule into the body.

According to some embodiments of the invention, the substance is included in the capsule.

According to some embodiments of the invention, the moving the capsule is performed through one or more body channels. According to some embodiments of the invention, the body channel is a blood vessel. According to some embodiments of the invention, the body channel is a lymph channel. According to some embodiments of the invention, the body channel includes Cerebro-Spinal Fluid.

According to some embodiments of the invention, the moving the capsule is performed through brain tissue.

According to some embodiments of the invention, the capsule is connected to an electric wire and the moving the capsule to the target location brings the end of the electric wire connected to the capsule to the target location.

According to an aspect of some embodiments of the present invention there is provided a method of measuring a velocity of flow of a fluid at a target location within a subject body including inserting a capsule including a locomotion element and a gamma emitting radioactive source into the body, using a radiation tracking subsystem to locate the gamma emitting radioactive source in three dimensions, moving the capsule to the target location within the body using a locomotion control subsystem which controls movement of the capsule by effecting movement of the locomotion element, based, at least partly, on location of the gamma emitting radioactive source provided by the radiation tracking subsystem, and measuring the velocity of flow of the fluid at the target location.

Further according to some embodiments of the invention, including, after the moving the capsule, releasing a therapeutic substance at the target location.

According to some embodiments of the invention, the locomotion control subsystem uses location information from the radiation tracking subsystem to automatically control movement of the capsule to the target location.

According to some embodiments of the invention, the locomotion control subsystem automatically controls movement of the capsule from a first location in the subject body to the target location.

According to some embodiments of the invention, the locomotion control subsystem controls movement of the capsule to the target location based, at least partly, on a three dimensional angiographic dataset.

Further according to some embodiments of the invention, including an optical tracker capable of monitoring the subject body and providing data for translating coordinates provided by the radiation tracking subsystem to coordinates provided by the three dimensional angiographic dataset.

According to some embodiments of the invention, the radioactive source emits gamma rays with an activity between 0.001 mCi and 0.5 mCi.

Further according to some embodiments of the invention, releasing a radioactive substance from the capsule into the fluid, using the radiation tracking subsystem to measure dispersal of the radioactive substance, measuring a time taken for the radioactive substance to disperse, and calculating a velocity of flow based, at least partly, on the time and the dispersal.

Further according to some embodiments of the invention, releasing a substance from the capsule into the fluid, measuring changes in impedance at the capsule, at two or more different times after the release of the substance, and calculating a velocity of flow based, at least partly, on the measuring of the changes in impedance and on the times the changes in impedance were measured.

Further according to some embodiments of the invention, releasing a substance from the capsule into the fluid, measuring changes in temperature at the capsule, at two or more different times after the release of the substance, and calculating a velocity of flow based, at least partly, on the measuring of the changes in temperature and on the times the changes in temperature were measured.

According to some embodiments of the invention, the moving the capsule is performed through one or more body channels. According to some embodiments of the invention, the body channel is a blood vessel. According to some embodiments of the invention, the body channel is a lymph channel. According to some embodiments of the invention, the body channel includes Cerebro-Spinal Fluid. According to some embodiments of the invention, the moving the capsule is performed through brain tissue.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings, identical structures, elements, or parts which appear in more than one drawing are generally labeled with the same numeral in all the drawings in which they appear. Dimensions of components and features shown in the drawings are chosen for convenience and clarity of presentation and are not necessarily shown to scale.

In the drawings:

FIG. 1 is a simplified pictorial illustration of an intra-body drug delivery system constructed and operative in accordance with an exemplary embodiment of the present invention;

FIG. 2 is a simplified flowchart illustration of a method for intra-body drug delivery in accordance with an exemplary embodiment of the present invention;

FIG. 3 illustrates the system of FIG. 1 in greater detail;

FIG. 4 is a simplified pictorial illustration of a Micro-Vascular-Capsule (MVC) and a flexible tube within an artery according to the system of FIG. 1;

FIG. 5 is a simplified pictorial illustration of a locomotion control unit according to the system of FIG. 1; and

FIG. 6 is a simplified flowchart illustration of a method for measuring fluid flow velocity in accordance with an exemplary embodiment of the present invention.

### DESCRIPTION OF PREFERRED EMBODIMENTS

Embodiments of the present invention comprise a system and method for placement of an object at an appropriate location within a body. More particularly, but not exclusively, a system and method for placement of an intra-body substance delivery mechanism at an appropriate location within a body channel, and for measurement of body fluid flow using the intra-body substance delivery system. The invention further comprises an automatic system and method for placement of the object at an appropriate location within the body.

The principles and operation of a system and method according to the present invention may be better understood with reference to the drawings and accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

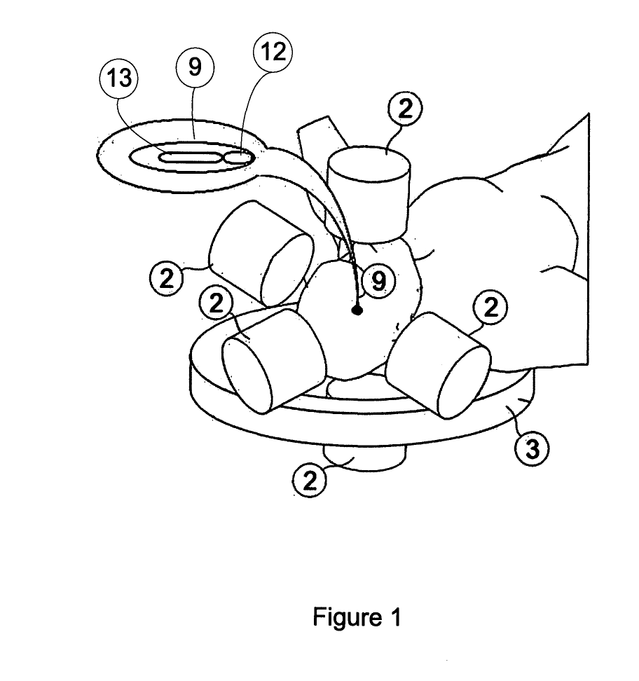

Reference is now made to FIG. 1, which is a simplified pictorial illustration of an intra-body drug delivery system constructed and operative in accordance with an exemplary embodiment of the present invention.

In the embodiment of FIG. 1, a Micro-Vascular-Capsule (MVC) 9 is inserted into a blood vessel (not shown) in a subject's body, for example, in the subject's head. The MVC 9 comprises a locomotion element 13, and a gamma emitting radioactive source 12.

A radiation tracking subsystem 3, capable of locating the gamma emitting radioactive source 12 in three dimensions, tracks the gamma emitting radioactive source 12, thereby tracking the MVC 9.

A locomotion control subsystem 2 controls the movement of the locomotion element 13, based on the location of the gamma emitting radioactive source 12 and the desired target location.

The radioactive source 12 is used for real time three-dimensional position tracking, which is performed by tracking sensors of the radiation tracking subsystem 3. Tracking data calculated by the radiation tracking subsystem 3 is passed in real time to the locomotion control subsystem 2 to provide positional feedback for a steering subsystem.

For example, the locomotion control subsystem 2 comprises electromagnets placed around a head of the subject, such that current in each of the electromagnets allows a combined magnetic field of the electromagnets to provide a necessary magnetic field and magnetic field gradient at a location of the MVC 9 to orient the MVC 9 and propel the MVC 9 in a desired direction. The orientation is provided by a magnetic field applying rotational torque on the locomotion element 13, and the propelling is provided by the magnetic field gradient applying force on the locomotion element 13.

Optionally alternative methods for propelling the MVC 9 are used. For example, the MVC 9 is tethered by means of a flexible tether which is anchored outside a patient's body, inserted into a blood vessel, and allowed to be propelled by the blood flow. By controlling how much of the tether is released, movement of the MVC 9 is controlled. The locomotion control subsystem 2 may use external magnets to provide steering of the MVC 9, by controlling orientation of the locomotion element 13, thereby controlling orientation of the MVC 9.

The flexible tether may optionally comprise a flexible tube, which can optionally serve for delivering the drug at the appropriate time. In exemplary embodiments of the present invention the MVC 9 comprises a substance delivery mechanism (not shown).

In some exemplary embodiments of the present invention the substance delivery mechanism is optionally a volume within the MVC 9 in which a substance to be delivered is carried.

In some exemplary embodiments of the present invention the substance delivery mechanism is a flexible tube connected to the MVC 9 through which a substance to be released is delivered.

When a flexible tether is used, the substance delivery mechanism optionally comprised in the MVC 9 may, or may not, comprise volume within the MVC 9 for storing the substance to be delivered.

Some of the substance to be delivered may be stored within the MVC 9 and some delivered by the flexible tube; all the substance may be stored within the MVC 9; all the substance may be delivered through the flexible tube; one substance may be delivered through the flexible tube and another substance may be stored in the MVC 9; and so on.

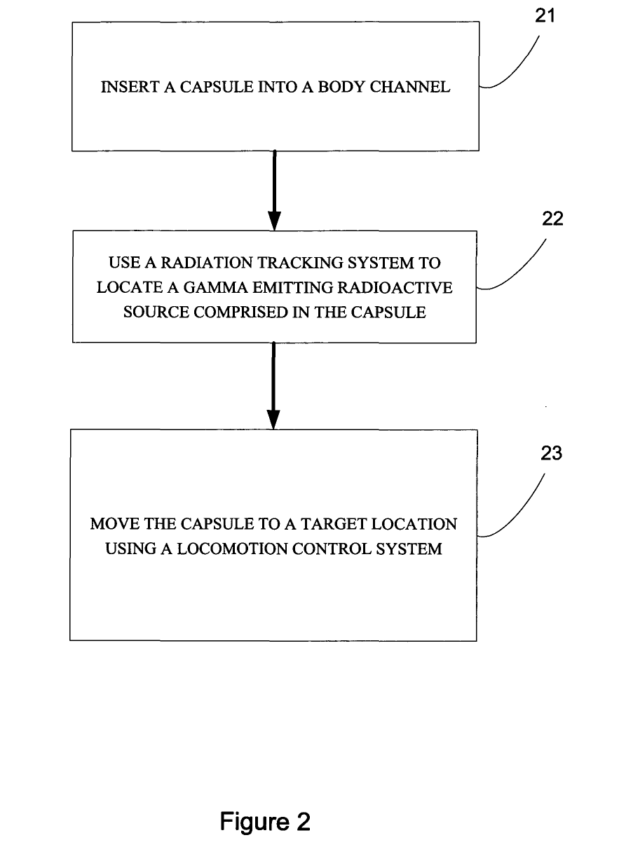

Reference is now made to FIG. 2, which is a simplified flowchart illustration of a method for intra-body drug delivery in accordance with an exemplary embodiment of the present invention.

The simplified flowchart illustrates the process for delivering a substance to a target location in a blood vessel within a subject body.

First, an MVC 9 comprising a locomotion element 13 and a gamma emitting radioactive source 12 is inserted into a blood vessel (stage 21).

Next, a radiation tracking subsystem 3 is used to locate the gamma emitting radioactive source 12 which is comprised in the MVC 9, in three dimensions (stage 22).

Finally, the MVC 9 is moved to the target location within the blood vessel. The move is effected using a locomotion control subsystem 2 which controls movement of the locomotion element 13 of the MVC 9. The locomotion control subsystem 2 controls the location of the MVC 9 based on location of the gamma emitting radioactive source 12 provided, in real time, by the radiation tracking subsystem 3 (stage 23).

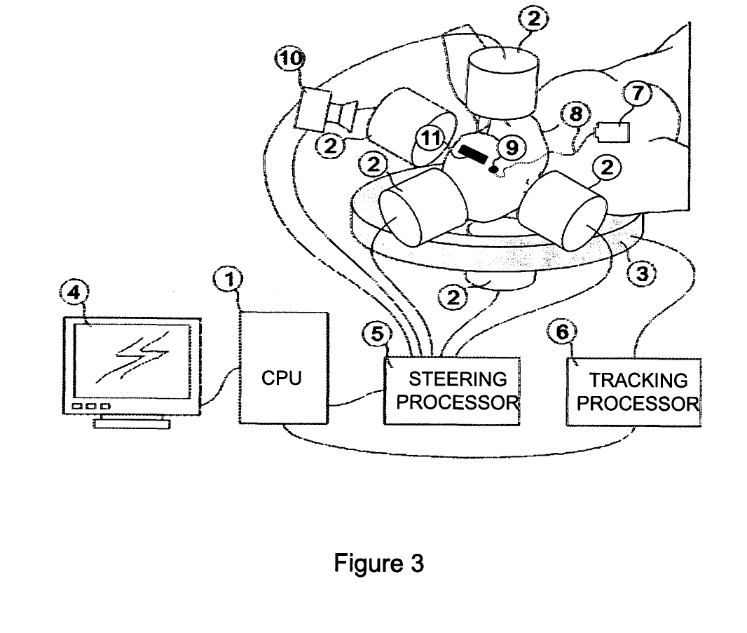

Reference is now made to FIG. 3, which illustrates the system of FIG. 1 in greater detail.

FIG. 3 shows a diagram of one possible embodiment of the intra-body drug delivery system. In this embodiment a Central Processor Unit (CPU) 1 receives three-dimensional angiographic data from a CT, MRI, or 3D-Angio exam. The angiographic data has preferably been prepared so that blood vessels are sharply contrasted to other organs, and thus easily picked out manually by operators, or automatically by the intra-body drug delivery system. Such preparation is done, for example, by dye injection of a contrasting dye during angiography.

An optical tracker 10 is mounted to observe a patient's head, and one or more optical markers 11 are located on the patient's head to enable registration of the patient's present head orientation and position with respect to the orientation and position of the angiographic data. The MVC 9, which is inserted into an artery and tethered by a flexible tube 8 to a locomotion control unit 7 outside the patient's body, contains a radioactive source 12 (FIG. 1) and locomotion element 13 (FIG. 1) comprising a magnet. The radioactive source 12 is used for real-time three-dimensional position tracking, which is performed by sensors of the radiation tracking subsystem 3 and tracking processor 6. Tracking data calculated by the tracking processor 6 is passed in real time to the CPU 1 to provide positional feedback for a steering subsystem of the locomotion control subsystem 2. Electromagnets placed around the head of the patient are driven by the steering processor 5 so that the current in each of electromagnets is such that their combined field provides the necessary field and field gradient at the location of the MVC 9 in order to orient the MVC 9 and propel the MVC 9 in a desired direction.

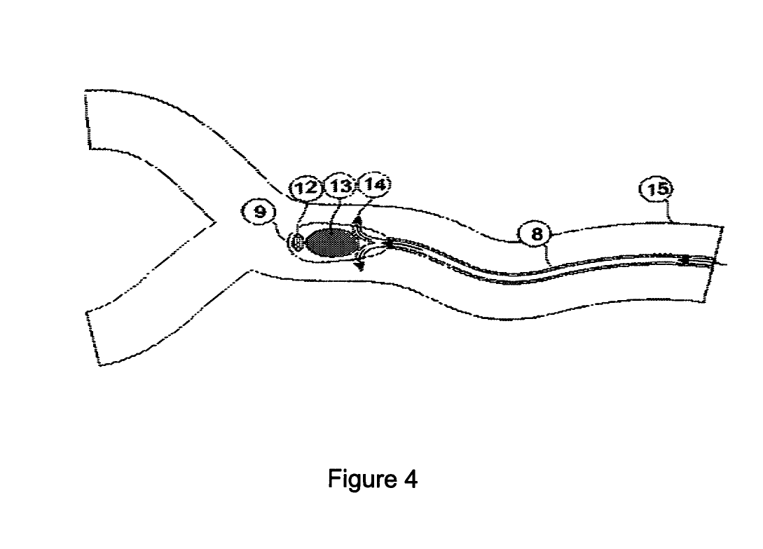

Reference is now made to FIG. 4, which is a simplified pictorial illustration of a Micro-Vascular-Capsule (MVC) 9 and a flexible tube 8 within an artery according to the system of FIG. 1.

FIG. 4 shows a diagram of one possible embodiment of the MVC 9 with an attached flexible tube 8. In this embodiment, the MVC 9 contains a radioactive source 12 and a permanent magnet as a locomotion element 13. The MVC is connected to the flexible tube 8 which is used both as a mechanical tether to control the locomotion of the MVC as it is propelled along by the blood flow, and as a tube through which drugs 14 can be delivered to the target artery 15.

The permanent magnet of the locomotion element 13 can be produced from a variety of magnetic materials, such as, by way of a non limiting example, paramagnetic materials, Ferro-magnets, ceramic magnets, and so on.

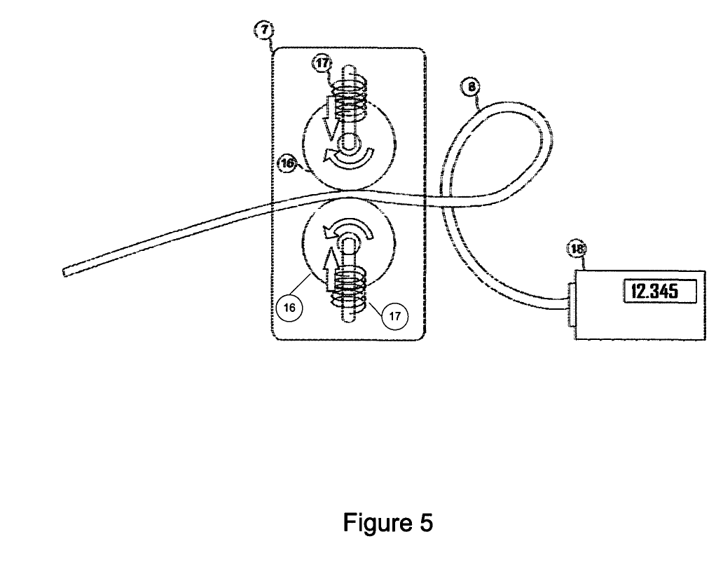

Reference is now made to FIG. 5, which is a simplified pictorial illustration of a locomotion control unit 7 (of FIG. 3) according to the system of FIG. 1.

In this embodiment; the locomotion control unit 7 contains motorized friction wheels 16 which control the movement of a flexible tube 8. The motorized friction wheels 16 are mounted on springs 17 such that the friction wheels 16 exert enough pressure on the flexible tube 8 to control its movement through the friction wheels 16. When fluid inside the flexible tube 8 is under sufficient pressure, the pressure separates the friction wheels 16 slightly, and the fluid flows past the friction wheels 16. The friction wheels 16 still press against the walls of the flexible tube 8 with enough force to control its movement. An electronically controlled pump 18 applies sufficient pressure to the fluid being pumped through the flexible tube 8 to enable the fluid to flow past the friction wheels 16.

Use of the above embodiments is now described by way of example with reference to treatment of stroke. Stroke treatment is chosen because it highlights certain advantages, such as the small size of the radioactive source 12 which is a tracked element, and the ease with which the MVC 9 can be navigated through complex vascular anatomy. However, this description is not intended to limit the invention in any way. The present embodiments can be used for intra-body drug delivery in many parts of the anatomy, not only in the brain, and not only for treatment of stroke. The embodiments can also be used with or without an attached flexible tube 8.

Treatment Process Description

Treatment of stroke typically begins with a CT exam of a patient's head. The CT enables a localization of a thrombosis and an assessment of resulting damage. If there is reason to believe that intra-body application of a thrombolytic agent using the MVC 9 is indicated, then a registration device, as further described below, may be used before, during, or after the CT scan, in order to enable registration with the automatic intra-body drug delivery system. A full head angiography dataset is produced as part of the CT exam, in addition to the standard stroke assessment images. The angiography dataset is used to produce a 3D roadmap of the vasculature, for guiding the MVC 9 within the brain.

After the CT exam, the patient is moved out of the CT system and the automatic intra-body drug delivery system is moved into place. The patient remains on the same patient bed. The registration device is then used to align the automatic intra-body drug delivery system with the angiography data which was obtained from the CT exam. The registration compensates for head movement which might take place between the CT exam and the MVC procedure, and during the MVC procedure.

Trained medical staff identify a location of the thrombosis on the 3D roadmap, and the automatic intra-body drug delivery system calculates and determines a vascular route from, for example, the carotid artery, to a treatment location.

Alternatively, if the medical staff prefer, they can manually determine the route from the carotid artery to the treatment location.

The MVC 9, with the attached flexible tube 8, are then placed into the carotid artery, and are steered and propelled along the determined vascular route by the locomotion control subsystem 2 under the guidance of the real time radiation tracking subsystem 3 and the CT-based 3D vascular roadmap.

Once the MVC 9 is in the treatment location, another CT exam may be performed to confirm the location of the MVC before drug delivery is initiated.

Thrombolytic drug delivery is initiated through the attached flexible tube 8 which the MVC 9 pulled through the vasculature to the treatment location. Treatment is monitored by testing blood flow in the artery at regular intervals during the thrombolytic therapy. When blood flow through the artery has returned, thrombolytic therapy is terminated. Optionally another CT exam may be performed to confirm the results of the treatment.

The MVC 9 is then removed by pulling out the flexible tube 8.

Exemplary Registration Mechanisms

For purpose of registering the coordinate system of the automatic intra-body drug delivery system to the coordinate system of the CT angiography database, it is necessary to either fix the head of the patient so that it can not move, or use a registration system to compensate for head movement. In order to make the automatic intra-body drug delivery system convenient to use and not require head fixation, a patient friendly registration system may be used.

An exemplary registration system for this application is an optical system. There are a number of types of optical systems which are commonly used for similar purposes. They include laser scanning systems which scan facial features of a patient and use the facial features to align the patient's head with the CT images; camera systems which image a pattern which is projected onto the body and analyze the projected image to produce a surface map, which is then compared to a surface map from the CT; and systems which optically track fiducial markers which are mounted on a patient's head. Alternatively, registration can be performed by manually locating a number of facial landmarks both in the CT images and on the patient upon beginning the MVC treatment.

One advantage of optical tracking of fiducial markers or camera-based body surface mapping over the use of facial landmarks and laser scanning is that they can be performed continuously throughout the treatment, compensating for head movement during the treatment.

Exemplary Insertion into the Carotid Artery

There are a number of ways in which the MVC 9 can be inserted into the carotid artery which could potentially reduce the need for an expert interventional radiologist and fluoroscopic guidance. Each of the ways has advantages and disadvantages, and trained medical staff using the automatic intra-body drug delivery system will need to decide, based on their personal preference and the condition of the patient, which is a best approach in each case.

Good approaches include a transcutaneous or open incision insertion directly into the carotid artery in the lower neck, and a brachial or axillary transcutaneous insertion. In the case of a brachial or axillary transcutaneous insertion, the MVC 9 is inserted inside a catheter until near the base of the carotid artery, at which point the MVC is released from the catheter and the external magnetic field propels the MVC 9 into the carotid artery. In this case the radiation tracking subsystem 3 is used to guide the insertion of the catheter, by tracking the MVC 9 which is in the tip of the catheter, and the radiation tracking subsystem 3 provides a display of the catheter tip position either on a user interface screen overlaid on a real or simulated anatomical image, or directly on the patient using a light projector or laser beam. The vasculature can also be projected onto the patient, based on the CT exam, to aid in catheter guidance. This approach requires that CT images of the upper chest region be acquired during the CT exam, for use in guiding catheter insertion.

An Exemplary MVC Propulsion and Steering Method

An exemplary steering and propulsion method for the stroke application is the use of a permanent magnet or a Ferro-magnet within the MVC 9 and a number of external electromagnets mounted around the patient's head. A superposition of magnetic fields from the electromagnets creates a magnetic field and a magnetic field gradient in a desired direction. By adjusting currents in the electromagnets, an appropriate magnetic field is constructed to steer and propel the MVC 9 in a desired direction. If the MVC 9 is tethered by the flexible tube 8, the flexible tube 8 is used as a mechanical restraint. Release of the mechanical restraint is controlled automatically by an electronically controlled motorized mechanism. The blood flow in the arteries is then used as the primary locomotive force, and the magnets perform primarily a steering function to select the correct vascular branch at each intersection.

Other configurations of external magnets, such as permanent magnets which are moved around in order to modify the magnetic field at the location of the MVC 9 or fixed permanent magnets with movable magnetic field shaping Ferro-magnets, are also contemplated.

The locomotion control unit 7 which releases or pulls the flexible tube 8 is designed to control the movement of the flexible tube 8 while allowing fluid to be pumped through the flexible tube 8. There are several designs which provide both of these functions.

One such exemplary design is shown in FIG. 5. This design has a pair of friction wheels 16 mounted on springs 17 grabbing the outside of the flexible tube 8 with enough force to control its movement, but such that a fluid under sufficient pressure within the flexible tube 8 can push the wheels apart and flow through. Using an estimate of a peak systolic blood pressure of 130 mmHg as a maximum pressure which can be applied to the capsule within the arteries, and a 6 mm diameter for the carotid artery, the resulting force on the capsule is 0.5 N. Using a conservative estimate of a coefficient of static friction between the friction wheels 16 and the flexible tube 8 of 0.5, the force with which the friction wheels 16 need to be pushed against the flexible tube 8 in order to control its movement is 1 N. The pressure inside the flexible tube 8 necessary to push back against the friction wheels 16 with 1N depends upon the size of the contact area between the flexible tube 8 and the friction wheels 16, which in turn depends on the diameter of the friction wheels 16.

Using the maximum pressure of a standard high pressure angioplasty balloon, 2×10⁶ N/m², as an estimate of the maximum internal pressure within the flexible tube 8, the contact area between the flexible tube 8 and the friction wheels 16 needs to be at least 0.5 mm² in order for the pressure in the flexible tube 8 to open the friction wheels 16. If the diameter of the flexible tube 8 is 0.5 mm, then the friction wheels need to be about 2 cm in diameter in order to have 0.5 mm² of contact area with the flexible tube 8. Using larger diameter friction wheels 16 increases the contact area with the flexible tube 8, thereby allowing the friction wheels 16 to be opened with lower pressure in the flexible tube 8.

Another design option which requires lower internal pressure to flow past the friction wheels 16 is to make a groove in the friction wheels 16, so that there is a location along the width of the friction wheel 16 where the friction wheel 16 is not pressing against the flexible tube 8. At that location very little pressure is required for the fluid to stretch the flexible tube 8 open slightly and flow past the friction wheels 16.

An Exemplary MVC Tracking Method

An exemplary tracking method for the MVC 9 is radiation tracking as described in Published PCT application WO 2006/016368 and in Published PCT application WO 2007/017846. The advantages of this tracking method over other known catheter tip tracking methods for this particular application are the small size, 0.01 mm³, of the tracked element which is the radioactive source 12, and the immunity to interference from metal objects and electric and magnetic fields.

An Exemplary Method of Electrode Placement within a Brain

An exemplary method of placement of an electrode within a brain involves inserting an MVC 9 comprising a magnet and a radioactive source 12 connected to a flexible wire comprising an electrode into a patient's head. The radiation tracking subsystem 3 is used to track the MVC 9. The locomotion control subsystem 2 provides magnetic propulsion to the MVC 9 in order to drag the electrode and the flexible wire through the brain tissue, as described above with reference to the exemplary MVC propulsion and steering method.

An Exemplary Flow Measurement Method

An exemplary flow measurement method for this application involves the assessment of dispersion time of a small volume of injected solution. The dispersion time is determined by whether fluid is blocked from flowing in the blood vessel in which the dispersion is assessed, and by a velocity of the fluid flow. The dispersion time of the solution can be measured in a number of ways, such as, for example, by injecting a solution with a temperature higher or lower than body temperature and measuring temperature over time; or a salt solution can be injected and impedance measured over time; or a radioactive solution can be injected and radioactivity dispersal measured over time.

One significant advantage of the radioactive liquid dispersion method is that the measurement can be made by the same external sensors used by the radiation tracking subsystem 3, instead of by sensors on the MVC 9, and the MVC 9 does not need to transmit data. The MVC can therefore be wireless.

A flow monitoring subsystem which is based on sensors on the MVC 9 requires transmission of sensed data to the outside of the body either via a wire or via wireless data transmission.

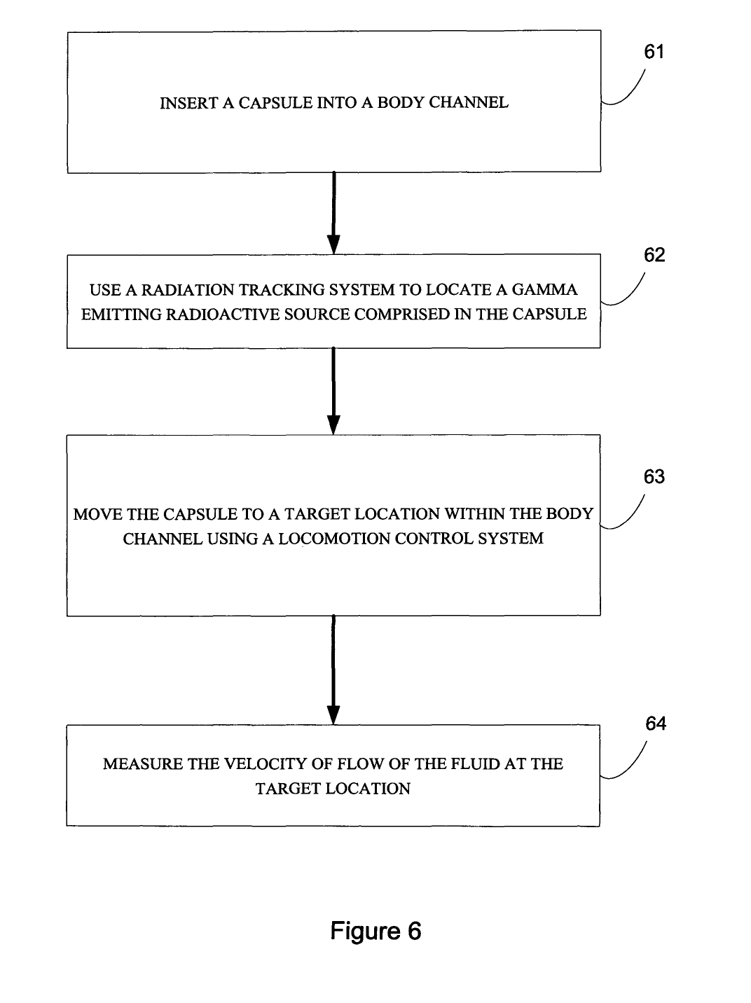

Reference is now made to FIG. 6, which is a simplified flowchart illustration of a method for measuring fluid flow velocity in accordance with an exemplary embodiment of the present invention. Reference is additionally made to FIG. 1.

The simplified flowchart illustrates the process for measuring a velocity of flow of a fluid at a target location in a blood vessel within a subject body.

First, a capsule, the MVC 9 of FIG. 1, comprising a locomotion element 13, and a gamma emitting radioactive source 12 is inserted into the blood vessel (stage 61).

Next, a radiation tracking subsystem 3 is used to locate the gamma emitting radioactive source 12 in three dimensions (stage 62).

Next, the capsule is moved to the target location within the blood vessel, using a locomotion control subsystem 2 which controls movement of the capsule by effecting movement of the locomotion element 13 (FIG. 1), based at least partly, on location of the gamma emitting radioactive source 12 provided by the radiation tracking subsystem 3 (stage 63).

Finally, the velocity of flow of the fluid at the target location is measured (stage 64).

In an exemplary embodiment of the present invention, the velocity of flow of the fluid at the target location is measured using the radiation tracking subsystem 3. When the MVC 9 reaches the target location, that is, after stage 63, the MVC 9 releases a radioactive substance. The radiation tracking subsystem 3 is used to measure an extent of dispersal of the radioactive substance. Based on a time taken for the radioactive substance to disperse, and the extent of the dispersal, a velocity of flow is calculated.

The radiation tracking subsystem 3 is capable of measuring an amount of radiation emitted by a radioactive source. While tracking, the tracker outputs a position of the radioactive source. To calculate fluid flow velocity, the radiation tracking subsystem 3 is locked onto a location of the radioactive source, and the MVC 9 releases a radioactive material. The radiation tracking subsystem 3 monitors the amount of radiation emitted from the location upon which it is locked. If the radioactive material disperses due to the flow of the medium, the amount of radiation emitted from the location will decrease over time, in a manner indicative of the flow velocity.

In some exemplary embodiments of the present invention, the radioactive material used to measure the flow velocity is the same type of material as used to track the MVC 9.

In other exemplary embodiments of the present invention, the radioactive material used to measure the flow velocity is a different material than used to track the MVC 9, such as, for example, a radioactive material which emits gamma rays of a different energy.

In another exemplary embodiment of the present invention, when the MVC 9 reaches the target location, that is, after stage 63, the MVC 9 releases a substance possessing electrical impedance different from that of the surrounding fluid. The velocity of flow of the fluid at the target location is calculated by measuring changes in impedance, at two or more different times after the release of the substance. The flow velocity is calculated based on the measuring of the changes in impedance and on the times the changes in impedance were measured.

In another exemplary embodiment of the present invention, when the MVC 9 reaches the target location, that is, after stage 63, the MVC 9 releases a substance possessing temperature different from that of the surrounding fluid. The velocity of flow of the fluid at the target location is calculated by measuring changes in temperature, at two or more different times after the release of the substance. The flow velocity is calculated based on the measuring of the changes in temperature and on the times the changes in temperature were measured.

In some exemplary embodiments of the present invention, when the capsule reaches the target location, that is, after stage 63, a dose of therapeutic substance is released.

Degree of Radioactivity and Half Life Considerations

An optimal degree of radioactivity and half-life for the radioactive source 12 used to track the MVC 9 depends on the application. In particular the degree of radioactivity and half-life depends on an amount of time that the MVC 9 remains within a patient's body. For the stroke application, the MVC 9 remains in the body for a relatively short period of time, for example on the order of 0.5-4 hours. It is therefore possible to use a radioactive source 12 with a short half-life, on the order of hours to days, making logistics of radioactive waste removal easier. In addition, the short period of time that the radioactive source 12 is in the body allows the use of a higher level of radioactivity, 100-300 uCi, which makes tracking more accurate.

For applications in which the radioactive source 12 must remain at the treatment site for days or even weeks, it is advantageous to reduce the level of radioactivity and to use a radioactive source 12 with a half-life on the order of weeks. In this case, it is advantageous to use a lower level of radioactivity in order to reduce the accumulated dose to the patient.

Safety

The level of radioactivity of the radioactive source 12 within the MVC 9 can be made low, for example below the above-mentioned range of 100-300 uCi, such that radioactivity will not cause any clinically significant damage to surrounding tissue.

Some gamma radiation sources also emit alpha radiation and/or beta radiation. In case of such gamma radiation sources, placing the radioactive source 12 at the center of the MVC 9 makes it safer by distancing the radioactive source 12, which also emits alpha and/or beta radiation, from the walls of the arteries within which it resides. Shielding from alpha and beta radiation is achieved by suitably thin layers of shielding material.

Safety of the magnetic steering and locomotion subsystem can be enhanced for the stroke application by using a head-only system instead of a full body system for two reasons: the heart is the most sensitive organ to rapidly changing magnetic fields, and the fields produced by a head-only system are small in the region of the heart; and since the magnets can be placed closer to the MVC 9 and can surround the MVC 9 from almost all directions, the magnetic fields that need to be produced by the external magnets in order to steer the MVC 9 are smaller than they would be in a whole body system.

It is expected that during the life of this patent many relevant devices and systems will be developed and the scope of the terms herein, particularly of the term Micro-Vascular-Capsule (MVC) is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for delivery of a capsule (9) to a target location within a subject body comprising:
a capsule, of a size less than 3 mm in length and less than 1 mm in diameter, comprising a locomotion element (13) and a gamma emitting radioactive source (12);
a real-time radioactive-radiation tracking subsystem (3) capable of locating the gamma emitting radioactive source (12) in three dimensions; and
a locomotion control subsystem (2) capable of controlling movement of the capsule (9) in real-time by effecting movement of the locomotion element (13), based, at least partly, on a location of the gamma emitting radioactive source (12) provided in real-time by the radiation tracking subsystem (3).

2. The system of claim 1 in which the locomotion control subsystem (2) is configured to use location information from the radiation tracking subsystem (3) to automatically control movement of the capsule (9) to the target location.

3. (canceled)

4. The system of claim 1 in which the locomotion control subsystem (2) controls movement of the capsule (9) to the target location based, at least partly, on a three dimensional angiographic dataset.

5. The system of claim 4 and further comprising an optical tracker (10) capable of monitoring the subject body and providing data for converting coordinates provided by the radiation tracking subsystem (3) to coordinates provided by the three dimensional angiographic dataset.

6. The system of claim 1 in which the radioactive source (12) emits gamma rays, the source (12) having an activity between 0.001 mCi and 0.5 mCi.

7. The system of claim 1 in which the gamma emitting radioactive source (12) occupies less than 10% of the capsule's (9) volume.

8. The system of claim 1 in which the locomotion element (13) in the capsule (9) comprises a magnetic material and the locomotion control subsystem (2) comprises a magnetic field configured to apply to the magnetic material in the capsule at least one member of the group consisting of a force and a torque.

9-11. (canceled)

12. The system of any one of the preceding claims and further comprising a substance delivery mechanism comprising a substance to be delivered and a release mechanism configured for releasing the substance.

13. (canceled)

14. The system of claim 12 and further configured to measure velocity of flow at the target location by releasing the substance at the target location and measuring dispersal of the substance at the target location, wherein the substance is radioactive, and the radiation tracking subsystem is configured:
to measure dispersal of the radioactive substance;
to measure a time taken for the radioactive substance to disperse; and
to calculate the velocity of flow based, at least partly, on the time and the dispersal.

15. (canceled)

16. The system of claim 14 and further configured to measure impedance at the capsule (9), at two or more different times after the release of the substance, and configured to calculate the velocity of flow based, at least partly, on the measured impedances and the times the impedances were measured.

17. (canceled)

18. The system of claim 12 in which the substance delivery mechanism comprises a tube which is connected to the capsule (9) through which a substance is delivered to the location of the capsule (9).

19. The system of claim 1 in which the locomotion control subsystem (2) includes a tether connected to the capsule (9).

20-22. (canceled)

23. The system of claim 19 in which the tether comprises at least one wire capable of conducting electrical current.

24. The system of claim 1 and wherein the system is configured to deliver the capsule (9) to the target location through one or more blood vessels.

25. (canceled)

26. The system of claim 24 in which the capsule (9) is configured to receive locomotion from blood flow and the locomotion control subsystem (2) is configured to provide steering to the capsule (9).

27-29. (canceled)

30. A method of delivering a capsule (9) to a target location within a subject body comprising:
inserting a capsule (9), of a size less than 3 mm in length and less than 1 mm in diameter, comprising a locomotion element (13) and a gamma emitting radioactive source (12) into the body;
using a real-time radioactive-radiation tracking subsystem (3) to locate the gamma emitting radioactive source (12) in three dimensions; and
moving the capsule (9) to the target location within the body using a locomotion control subsystem (2) to control movement of the capsule (9) in real-time by effecting movement of the locomotion element (13), based, at least partly, on location of the gamma emitting radioactive source (12) provided in real-time by the radiation tracking subsystem (3).

31. The method of claim 30 in which the locomotion control subsystem (2) uses location information from the radiation tracking subsystem (3) to automatically control movement of the capsule (9) to the target location.

32. (canceled)

33. The method of claim 30 in which the locomotion control subsystem (3) controls movement of the capsule (9) to the target location based, at least partly, on a three dimensional angiographic dataset.

34. The method of claim 33 and further comprising an optical tracker (10) capable of monitoring the subject body and providing data for converting coordinates provided by the radiation tracking subsystem (3) to coordinates provided by the three dimensional angiographic dataset.

35. The method of claim 30 in which the radioactive source (12) emits gamma rays with an activity between 0.001 mCi and 0.5 mCi.

36. The method of claim 30 in which the gamma emitting radioactive source (12) occupies less than 10% of the capsule's (9) volume.

37. The method of claim 30 and further releasing a substance from the capsule (9) into the body.

38. (canceled)

39. The method of 38 claim 30 in which the moving the capsule (9) is performed through one or more blood vessels.

40-43. (canceled)

44. The method of claim 30 in which the capsule (9) is connected to an electric wire and the moving the capsule (9) to the target location brings the end of the electric wire connected to the capsule to the target location.

45. A method of measuring a velocity of flow of a fluid at a target location within a subject body comprising:
inserting a capsule (9) comprising a locomotion element (13) and a gamma emitting radioactive source (12) into the body;
using a real-time radioactive-radiation tracking subsystem (3) to locate the gamma emitting radioactive source (12) in three dimensions;
moving the capsule (9) to the target location within the body using a locomotion control subsystem (2) which controls movement of the capsule (9) in real-time by effecting movement of the locomotion element (13), based, at least partly, on location of the gamma emitting radioactive source (12) provided in real-time by the radiation tracking subsystem (3); and
measuring the velocity of flow of the fluid at the target location.

46. The method of claim 45 and further comprising, after the moving the capsule (9), releasing a therapeutic substance at the target location.

47. The method of claim 45 in which the locomotion control subsystem (2) uses location information from the radiation tracking subsystem (3) to automatically control movement of the capsule (9) to the target location.

48. (canceled)

49. The method of claim 45 in which the locomotion control subsystem (2) controls movement of the capsule (9) to the target location based, at least partly, on a three dimensional angiographic dataset.

50. The method of claim 49 and further comprising an optical tracker (10) capable of monitoring the subject body and providing data for translating coordinates provided by the radiation tracking subsystem (3) to coordinates provided by the three dimensional angiographic dataset.

51. The method of claim 45 in which the radioactive source (12) emits gamma rays with an activity between 0.001 mCi and 0.5 mCi.

52. The method of claim 45 and further:
releasing a radioactive substance from the capsule (9) into the fluid;
using the radiation tracking subsystem (3) to measure dispersal of the radioactive substance;
measuring a time taken for the radioactive substance to disperse; and
calculating a velocity of flow based, at least partly, on the time and the dispersal.

53. The method of claim 45 and further:
releasing a substance from the capsule (9) into the fluid;
measuring changes in impedance at the capsule (9), at two or more different times after the release of the substance; and
calculating a velocity of flow based, at least partly, on the measuring of the changes in impedance and on the times the changes in impedance were measured.

54. The method of claim 45 and further:
releasing a substance from the capsule (9) into the fluid;
measuring changes in temperature at the capsule (9), at two or more different times after the release of the substance; and
calculating a velocity of flow based, at least partly, on the measuring of the changes in temperature and on the times the changes in temperature were measured.

55. The method of claim 45 in which the moving the capsule (9) is performed through one or more blood vessels.

56-59. (canceled)

60. The system of claim 1 in which the capsule (9) is of a size suitable for being injected into a body by a needle.
